(19) United States
(12) Patent Application Publication
(10) Pub. No.: US 2015/0351647 A1
(43) Pub. Date: Dec. 10, 2015

(54) EFFECTIVE CPR PROCEDURE WITH REAL TIME EVALUATION AND FEEDBACK USING SMARTPHONES

(71) Applicant: University of North Texas, Denton, TX (US)

(72) Inventors: Ramanamurthy Dantu, Richardson, TX (US); Neeraj Gupta, Allen, TX (US); Vishnu Dantu, Richardson, TX (US); Zachary Morgan, Sugar Land, TX (US)

(21) Appl. No.: 14/701,222

(22) Filed: Apr. 30, 2015

Related U.S. Application Data
(60) Provisional application No. 61/987,357, filed on May 1, 2014.

(51) Int. Cl. A61B 5/024; A61H 31/00; A61B 5/00

(52) U.S. Cl. CPC A61B 5/02438; A61B 5/02416; A61B 5/68; A61B 5/0022; A61H 31/00

(57) ABSTRACT

A method for performing CPR, comprising activating an application on one or more mobile phones having one or more sensors, placing one or more mobile phones on the finger of a subject to collect information about the subject, determining whether CPR is necessary based on the collected information about the subject, calibrating the sensors of the one or more mobile phones, placing the one or more mobile phones in a position on a hand of a user of the one or more mobile phones, administering chest compressions to the subject, activating a sensor of the one or more mobile phones, including an accelerometer sensor, to permit the application to capture information about the chest compression rate and displacement relating to movement of the chest of the subject, and transmitting the chest compression rate and displacement information of the subject to the emergency dispatcher using the mobile phone.

This application claims priority to U.S. Provisional Patent Application No. 61/987,357, entitled “Effective CPR Procedure with Real Time Evaluation and Feedback Using Smartphones,” the entire content of which is hereby incorporated by reference.

This invention was made with government support under Grant Nos. CNS-0751205, CNS-0821736, G72376, and G72373 awarded by the National Science Foundation. The United States Government has certain rights in the invention.

### BACKGROUND OF THE INVENTION

The present disclosure relates generally to effective CPR procedures, and more specifically to administering CPR procedures in connection with regulation devices which monitor and transmit information to a 9-1-1 operator who analyzes the information and relays real-time feedback and guidance about the optimum CPR procedures to administer.

Cardio Pulmonary Resuscitation (CPR) is an emergency procedure performed on people under cardiac arrest and on people who stop breathing due to reasons such as drowning. CPR's main benefit is that it maintains blood flow, which prevents tissue and brain damage. The procedure involves creating artificial blood circulation by applying rhythmic pressure to a person's chest. The blood then carries oxygen to body organs.

With nearly 600,000 deaths a year, heart disease is the leading cause of mortality in the United States accounting for approximately two thirds of deaths occurring from sudden cardiac arrest. Almost 600,000 cardiac arrests occur outside of a hospital setting each year with nearly 90 percent of these events occurring at home. Sudden cardiac arrest is the leading cause of death in adults with more than 1,000 sudden cardiac arrests occurring each day. In addition, death occurs in 90 percent of individuals experiencing a sudden cardiac arrest outside of a hospital setting. Guidelines from the American Heart Association has provided sufficient evidence to support the administration of cardio pulmonary resuscitation (CPR) and accessibility of automated external defibrillator to improve survival rates following cardiac arrest. In order to provide the highest quality CPR, the AHA puts priority on specific characteristics including compression fraction, rate, depth, and reduced leaning to ensure a full release (recoiling). Effective, high-quality CPR provided immediately following cardiac arrest can dramatically increase survival rates, however less than 30% of individuals experiencing an out-of-hospital cardiac arrest receive CPR from a bystander before emergency response personnel arrival. In many developing countries the survival rate falls further to few percentage.

The primary benefit of CPR is the maintenance of blood flow to vital organs, such as the brain, until more advanced medical procedures can be performed. Effective CPR provided immediately following cardiac arrest can double or triple survival rates, however only 32 percent of individuals experiencing a cardiac arrest receive CPR from a bystander. The almost 70 percent of individuals who fail to respond in a cardiac arrest situation, generally do not respond because they do not know how to administer CPR or do not feel comfortable with their technique if they have been trained. Due to the low response rate of bystanders to administer CPR to individuals suffering from cardiac arrest, technological innovation to improve CPR technique and provide real-time evaluation and feedback would be beneficial and could lead to increased cardiac arrest survival rates.

References to resuscitation attempts can be found in ancient texts that date back thousands of years, but the first known attempts at resuscitation in modern times occurred in the 18th century. Practitioners, then, used various techniques to resuscitate a person who was unconscious or not breathing. These included “blowing air” into the mouth, massaging the chest, tickling the throat, or applying manual pressure to the abdomen. These methods were most effective when used for drowning victims. Over the years, practitioners refined the techniques, and until 1950s, the accepted resuscitation method was applying back pressure and arm lifting. James Elam developed the currently used CPR method in 1954. He along with Dr. Peter Safar demonstrated the superiority of their CPR method to earlier methods. Their method used chest compressions in combination with periodic mouth-to-mouth breathing. Latest guidelines from the American Heart Association have modified the Elam and Safar CPR approach; the AHA recommends using Continuous Chest Compression (CCR) because this approach works better than periodically stopping compressions for mouth-to-mouth breathing.

The first organized attempt to make citizens a part of the emergency procedure in cases of cardiac arrest was made in Seattle in March of 1970. Fire department personnel were trained in CPR so that they could perform it on the victim before paramedics arrived to attempt defibrillation. The data gathered from this exercise proved that when CPR was started within 2-3 minutes of the event, survival chances increased. In 1972 the project was expanded to train over 100,000 people. Over the years, community-based CPR training of general public has expanded across the United States. In 1981, Washington State started a program to give telephone instructions for CPR. Emergency professionals learn to provide CPR instructions to the callers before the paramedics arrived. This increased the rate of bystander-provided CPR by over 50%.

Effective CPR

Effective CPR consists of the following procedure:
Lay the person flat on his back.
Place your hand flat on the person's upper chest between the nipples. For infants only two fingers are used—the middle finger and the index finger. For adults only, place your second hand above the first hand (for children only one hand is used).
Start applying pressure to compress the chest.
The recommended rate of chest compressions is about 100 per minute.

The depth of chest compression is about 2-2.5 inches for adults, about 1-1.5 inches for children, and about ⅓ inch for infants. Original AHA guidelines emphasize A-B-C as a CPR guideline. In the acronym, A stands for airways, meaning that the person giving CPR needs to make sure that the airways are open; B stands for Breathing, meaning that the person giving CPR does mouth-to-mouth breathing; and C stands for Chest Compressions. In traditional methods, periodic mouth-to-mouth breathing is also done to replenish oxygen supply, but newer guidelines suggest that continued compression is more important. The acronym has been modified to C-A-B. Consequently, mouth-to-mouth breathing has now become the third, optional, portion of CPR. The primary reason for the change is that most bystanders or paramedics hesitate to use mouth-to-mouth breathing with unknown people because mouth-to-mouth breathing may cause spread of infectious diseases. Apart from concerns over infections, there has also been discussion on how often to give mouth to mouth breathing. Normally there is enough oxygen in the blood stream to only do continuous chest compressions. Breathing is needed only if the oxygen saturation in the blood stream falls. Since an oximeter may not be available at that moment, there is no way to determine the oxygen level; therefore, it is difficult to determine whether mouth-to-mouth breathing is required. Making mouth-to-mouth breathing optional ensures that chest compression begins within the critical survival window.

Experts are also debating the need to give mouth-to-mouth breathing in cases where the blood oxygen saturation level falls. A person's Blood Oxygen Saturation Level (BOS) indicates how efficiently a body's blood cells retain oxygen. Cardiopulmonary Resuscitation is performed to force the movement of oxygenated blood through the circulatory system and prevent the damage of vital organs in the body. The level is measured by analyzing the ratio between the amount of oxygenated hemoglobin and the total amount of hemoglobin present. The ideal ratio ranges from 95% to 100%. Among other things, BOS level ranges can help to determine a person's risk of lung disease and tissue death. The BOS level's ability to determine the Cardiopulmonary Resuscitation's efficiency is discussed further herein. With the knowledge of a victim's blood oxygen saturation level, the decision to give mouth-to-mouth breathing may be necessary to keep oxygenation at a healthy level.

Use of Technology for Effective CPR

Over the years, awareness amongst the general public that CPR can be a lifesaving procedure has increased. There is a growing use of technology that aids people in performing CPR. Several devices provide CPR training. These devices improve the quality of CPR by providing feedback on proper placement of hands on chest and the correct frequency and depth of compressions. Mechanical devices which give accurate frequency and depth of chest compression provide automatic CPR. Studies have shown that these automated CPR devices improve the survivability of patients who need out-of-hospital CPR. During an emergency it is likely that a person trained in CPR may not be available. In such situations 9-1-1 operators help the caller to administer CPR by giving instructions over a phone. In such instances, a readily available technology would be useful in ensuring that people untrained could deliver CPR properly. Recently smartphone applications have provided video instructions on how to give CPR. If the application is not available, a 9-1-1 operator can help in downloading that application. However, having to download the application and then watch the video seriously reduces the window of survivability for the injured person. There is a need for a device that gives real-time feedback on the quality of CPR.

### BRIEF SUMMARY OF THE INVENTION

The present disclosure relates generally to effective CPR procedures, and more specifically to administering CPR procedures in connection with regulation devices which monitor and transmit information to a 9-1-1 operator who analyzes the information and relays real-time feedback and guidance about the optimum CPR procedures to administer.

During an emergency situation, it is highly likely that persons trained in CPR are unavailable. Even though devices can provide automatic CPR, these devices are highly unlikely to be accessible at the time of need. In such cases, an untrained person will need to administer CPR. In these situations, 9-1-1 operators provide CPR instructions over a phone. However, the success of such an approach depends upon the emotional and physical capabilities of the person actually administering the CPR. With newer technology, the operator may even attempt to send video instructions, which may assist in improving the CPR given. However, again, the 9-1-1 operator may not have all the information necessary to determine whether the CPR is being done efficiently and helping the injured person.

Currently, 9-1-1 operators cannot evaluate the results of CPR remotely and, in fact, there is no proven method to evaluate CPR effectiveness even when a trained person is giving the CPR. In this paper, we present a method to evaluate CPR performance in real-time without the need of special devices. Using the sensors in a smartphone, such as an accelerometer, this paper describes an application that can evaluate and guide a person in giving effective CPR while providing timely feedback to the 9-1-1 operator.

The two important CPR parameters are the frequency and depth of compressions. In the present disclosure, smartphones are used to calculate these factors and to give real-time guidance to improve CPR. In addition, effective smartphone application has been developed that can be used to provide real-time evaluation and feedback during CPR. Before CPR, the application can provide accurate pulse rate for the victim. During CPR, the smartphone can be attached above the hands of the person administering CPR and the smartphone application can calculate the frequency and depth of chest compressions with accuracy greater than 95 percent. In addition, the application can measure pulse rate through the use of the smartphone's camera lens. The smartphone application could be the first application available that provides real time feedback concerning chest compression, recoiling (leaning) during compression, and pulse rate. With a special glove and built-in electronics, the system can be used to automatically activate and connect to a remote hospital/physician. Although not necessarily recommended, the smartphone application could also be used to effectively administer CPR, even by people who have not been trained to administer CPR.

The application can also measure blood oxygen saturation levels through the use of the smartphone's camera lens to measure the visible and infrared spectrum of the oxyhemoglobin and de-oxy hemoglobin, respectively. The fingertip surface of the individual suffering from cardiac arrest can be placed on the smartphone's camera lens. By taking a video, a beam of near-infrared light is passed through the finger and the smartphone creates video of the area of analysis. The video is then analyzed to determine the red green blue (RGB) values. The RGB values are further analyzed for the scattering effect of near-infrared light. This scattering effect can be utilized to determine the blood oxygen saturation level. If blood oxygen saturation falls below an acceptable threshold, the person giving CPR can be asked to do mouth-to-mouth breathing. The 9-1-1 operator receives this information real time and can further guide the person giving CPR. Experiments show accuracy greater than 90% for compression frequency, depth, and oxygen saturation.

The developed technology can be used for effective CPR training, remote monitoring, evaluation and certification of training of community workers, resuscitation quality improvement (RQI) for healthcare workers, and potential use by bystanders during cardiac arrest. Since the low-cost smartphone-based application has remote monitoring capabilities, the technology could provide potential lifesaving support to individuals experiencing sudden cardiac arrest in economically deprived communities, developing countries.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a diagram of a CPR evaluation system in accordance with an exemplary embodiment of the present disclosure;

FIG. 2 shows a diagram of a CPR evaluation system in accordance with an exemplary embodiment of the present disclosure;

FIG. 3 shows a method of administering CPR in accordance with an exemplary embodiment of the present disclosure;

FIG. 4 shows a method of detecting the start of CPR administration in accordance with an exemplary embodiment of the present disclosure;

FIG. 5 shows a method of calculating frequency of CPR chest compressions in accordance with an exemplary embodiment of the present disclosure;

FIG. 6 shows a method of calculating frequency and depth of CPR chest compressions in accordance with an exemplary embodiment of the present disclosure;

FIG. 7 shows a method of resetting velocity during calculations of CPR compression depth in accordance with an exemplary embodiment of the present disclosure;

FIG. 8 shows a method of calculating depth of CPR chest compressions in accordance with an exemplary embodiment of the present disclosure;

FIG. 9 shows a diagrams of different stages of a method of administering CPR in accordance with an exemplary embodiment of the present disclosure;

FIG. 10 shows an accelerometer measurement plot in accordance with an exemplary embodiment of the present disclosure;

FIG. 11 shows an accelerometer measurement plot in accordance with an exemplary embodiment of the present disclosure;

FIG. 12 shows an accelerometer measurement plot in accordance with an exemplary embodiment of the present disclosure;

FIG. 13 shows an accelerometer measurement plot for compression depth during CPR in accordance with an exemplary embodiment of the present disclosure;

FIG. 14 shows an acceleration, velocity, and distance measurement plot during CPR in accordance with an exemplary embodiment of the present disclosure;

FIG. 15 shows a mannequin used to record measurements for CPR administered in accordance with an exemplary embodiment of the present disclosure;

FIG. 16 shows a scatter plot of the accuracy of each compression during CPR administered in accordance with an exemplary embodiment of the present disclosure;

FIG. 17 shows a bar graph of the calculated depth and actual depth of compressions during CPR administered in accordance with an exemplary embodiment of the present disclosure;

FIG. 18 shows a scatter plot of compression depth alerts during CPR administered in accordance with an exemplary embodiment of the present disclosure;

FIG. 19 shows an occlusion spectroscopy used to make measurements during CPR administered in accordance with an exemplary embodiment of the present disclosure;

FIG. 20 shows measurements of oxygen saturation levels using phone sensors in accordance with an exemplary embodiment of the present disclosure;

FIG. 21 shows the decay rate of oxygen for students measured using phone sensors in accordance with an exemplary embodiment of the present disclosure;

FIG. 22 shows an example of portions of a screen made available to a 911 operator as part of an operator portal in accordance with an exemplary embodiment of the present disclosure; and

FIG. 23 shows a method of administering hands only CPR in accordance with an exemplary embodiment of the present disclosure.

### DETAILED DESCRIPTION OF THE INVENTION

The present disclosure relates generally to effective CPR procedures, and more specifically to administering CPR procedures in connection with regulation devices which monitor and transmit information to a 9-1-1 operator who analyzes the information and relays real-time feedback and guidance about the optimum CPR procedures to administer.

In preferred embodiments, this disclosure relates to a method and application for providing CPR training using off the shelf mobile phones. The application provides stand alone real-time feedback of depth, frequency and recoil of compressions without using the Internet. It allows the users to record and evaluate the training sessions and to train large groups with a single desktop. It alerts the administrator of ineffective CPR. It monitors CPR and subsequently alerts the CPR administrator if there is more than 20% rest during administration. It provides periodic real-time audio feedback through alerts for improving an effective administration of compression frequency and depth, and allows for a dynamic change of frequency of alerts. The CPR metrics can be calibrated based on age and gender of the patient.

In additional preferred embodiments, the method and application include capabilities for remote support and monitoring, as well as stand-alone capabilities. Real-time feedback can be provided from a remote operator (e.g., 911 operator). Vital signs and compression logs can be transmitted to a remote location. Vital signs can be collected with multiple devices. The mobile phone's 3G, 4G, and WiFi capabilities can be utilized. The application may allow for a NG911 Operator Portal that can be accessed by the operator and used to monitor vital signs and the progress of CPR administration.

In certain embodiments, a special glove with built-in electronics can automatically activate the suitable application and connect to a preselected remote site such as a hospital or physician.

With regard to reporting, in certain embodiments the application can generate a report card after the CPR administration. The report card includes the following parameters: Chest compression fraction, compression frequency, compression depth, and leaning (recoil).

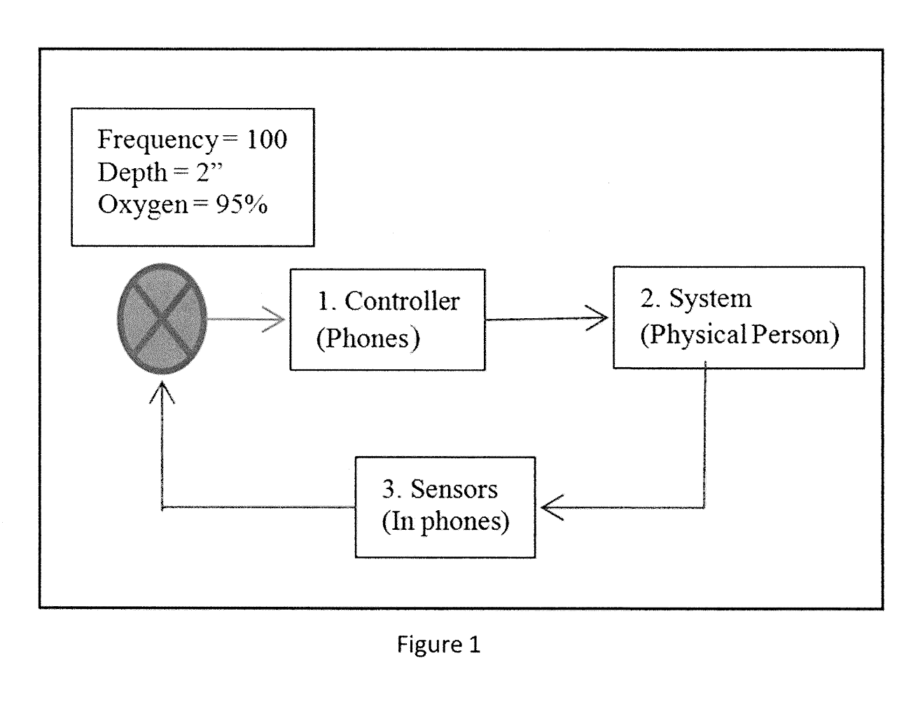

FIG. 1 depicts a flow chart of a CPR evaluation system. As depicted in FIG. 1, the smartphone's sensors provide feedback about chest compression frequency and depth, and about blood oxygen saturation levels.

Referring to FIG. 1, a smartphone controller (box 1) holds an algorithm to evaluate data from sensors (box 3). The system (box 2) consists of the affected person and the person giving the CPR. A feedback loop to the System (CPR giver) provides corrective action.

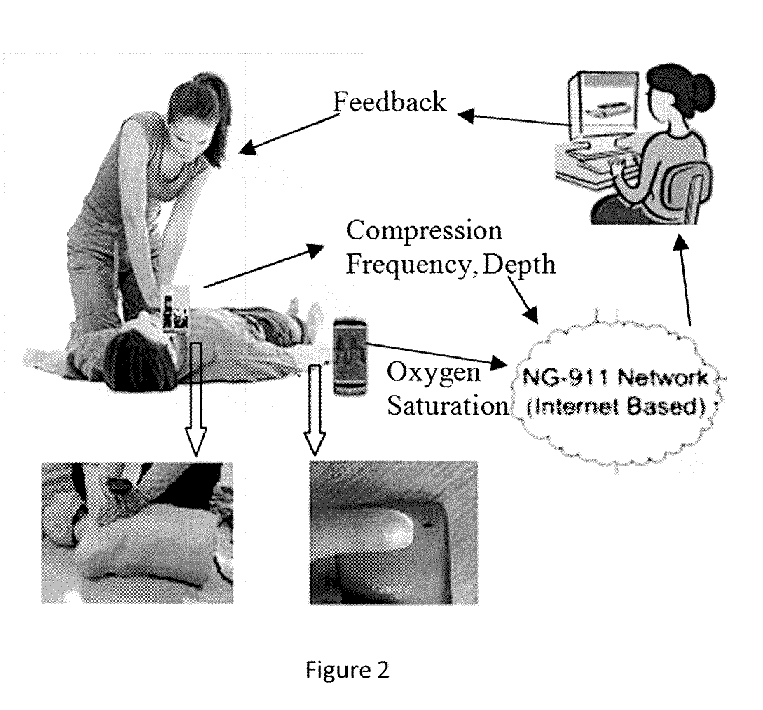

As mentioned in prior sections, continuous chest compressions have been emphasized over cardiopulmonary resuscitation as the most critical CPR procedure to perform in an emergency. That said, mouth-to-mouth breathing remains a viable option in certain cases, especially when trained personnel are present. In an exemplary embodiment, a smartphone application is presented which measurers the blood's oxygen saturation without specialized equipment. In conjunction, one smartphone can be used by the person giving the CPR where it measures the frequency and depth of compressions. A second smartphone can then be used to measure the oxygen saturation level. The data from these smartphones is continuously reported to the 9-1-1 operator who can use the information to guide the CPR giver. FIG. 2 summarizes how CPR could be enhanced with these devices.

Referring now to FIG. 2, the compression frequency, depth and the oxygen saturation levels are reported to the 9-1-1 operator via the smartphone device. The person giving CPR has a phone tied to her hands, as shown in the picture below the arrow, A smartphone is also placed near the hand of the person receiving the CPR with finger on the camera lens as shown in the second picture below the arrow.

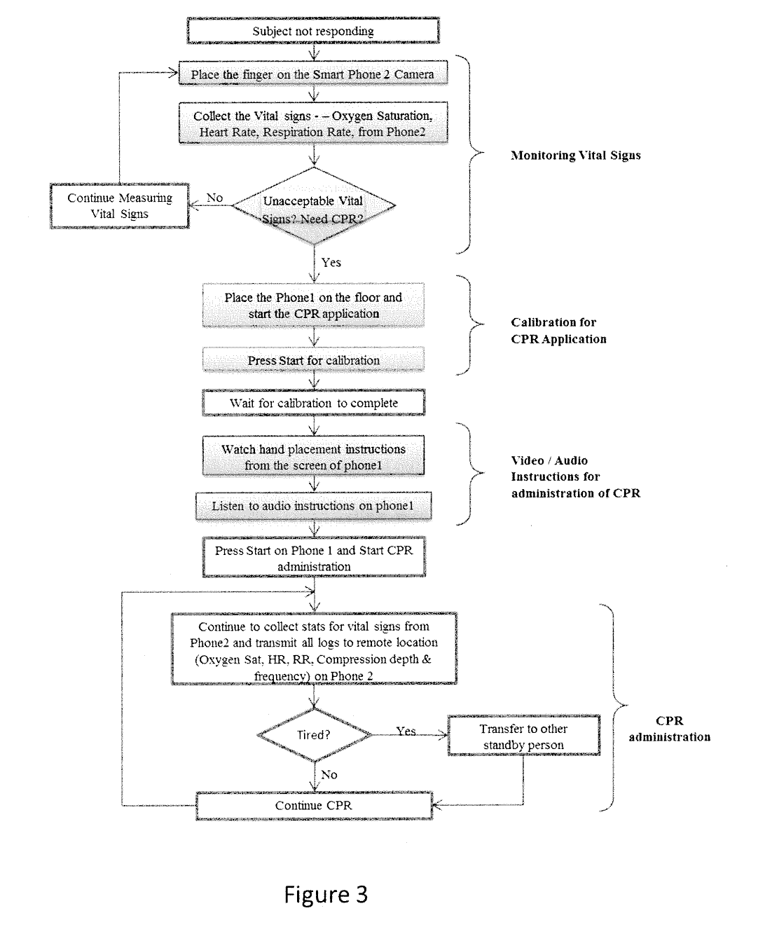

In one embodiment of the present disclosure, CPR is administered to unresponsive subjects or mannequins during CPR training using the algorithm shown in FIG. 3. A first smartphone equipped with sensors is placed on a finger of the subject. The sensors of the smartphone are used to collect vital signs of the subject including oxygen saturation levels, heart rate, respiration rate, and any other suitable vital signs. Based on the collected information during this monitoring stage, the smartphone with make a determination of whether CPR is necessary. These measured vital signs can be transmitted to a remote location for expert analysis, logged into the smartphone memory, or a combination thereof.

After the determination that CPR is necessary, the smartphone with prompt the user (i.e. CPR administrator) to calibrate the sensors of the smartphone, or optionally, to calibrate the sensors of a second smartphone. This calibration involves allowing the sensors (including accelerometers, cameras, and the like) of the one or more smartphones to calibrate to ground levels (i.e. extracting external “noise” from the environment which could disrupt the accuracy of measurements.)

In one embodiment of the present disclosure, the calibration progress can include the following steps:
Put the one or more smartphones on a flat surface, approximately parallel to the Earth
Start the application and push the “Calibrate” button
“NaturalGravity”=9.81 m/sec²
Repeat the following for 15 seconds
   Get the accelerometer reading “Ace”
   gravDeviation[i]=Acc−NaturalGravity
For each second Calculate statistical variation from the NaturalGravity
Calculate the noise Correction parameter based on the standard deviation.

Once the calibration stage is completed, the administration of CPR should begin. Specifically, the user will place one smartphone on the back of the user's hands. The smartphone may be held in place using any restraining device such as a belt, shoelaces, plastic bag, rubber band, tape, or any suitable alternative. Optionally, the user can leave the additional one or more smartphones on the finger of the CPR subject during this process to continue monitoring the vital levels of the subject.

In one embodiment of the present disclosure, the one or more smartphone can display audio, video, or audio/video instruction on how to administer CPR properly including hand placement, compression methods, compression frequency, compression depth, and the like. If only one smartphone is present, the user can periodically exchange the smartphone between positions to monitor vital signs on the subject's finger or to monitor compression measurements on the back of the user's hand.

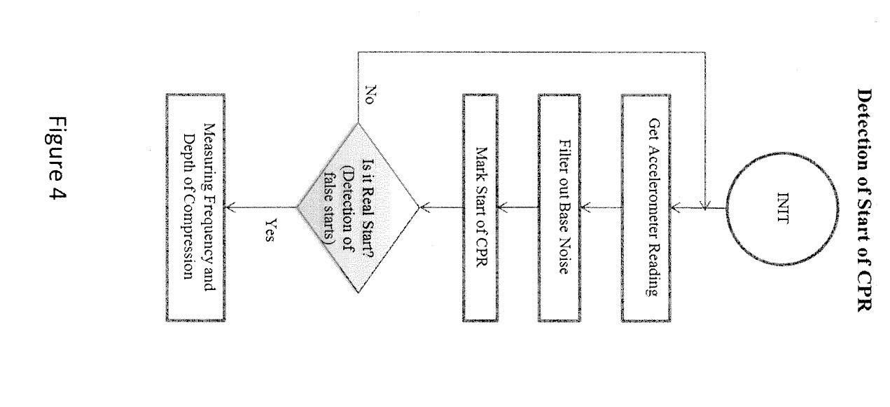

In one embodiment of the present disclosure, the sensors (including accelerometer) of the one or more smartphones are used to detect the start of CPR as shown in FIG. 4. In one embodiment of the present disclosure, the start of CPR administration can be detected as follows:
Get Accelerometer reading
Filter out calculated noise by subtracting the noise parameter
Repeat Step 1 & 2 for X readings // X is a parameter that can be set
If the readings show movement of the one or more smartphones in compression direction
   Positive accelerometer reading is compression which indicates the start of CPR administration
   Negative accelerometer reading is recoil
Else go back to Step 1.

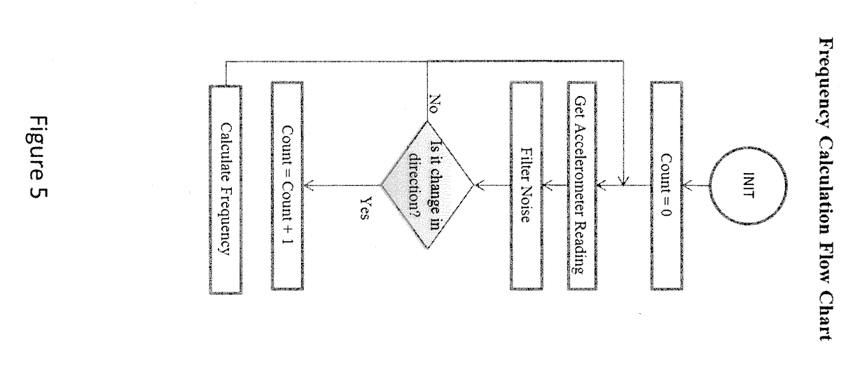

In one embodiment of the present disclosure, the sensors (including accelerometer) of the one or more smartphones are used to calculate the frequency of compressions during CPR as shown in FIG. 5.
Set count=0
Set time of start=0
Get Accelerometer reading
Filter out Calculated noise by subtracting the noise parameter
Repeat step 1 & 2 for Y readings // Y parameter may have a different value than X
If the movement direction shows change then increase the count by 1
   Positive accelerometer reading is compression,
   Negative reading is recoil
Calculate time passed from previous reading (timePassed)
Increment time by timePassed
If time is 1 second then frequency=count
   Reset time=0
   Reset count=0
Else repeat Step 1.

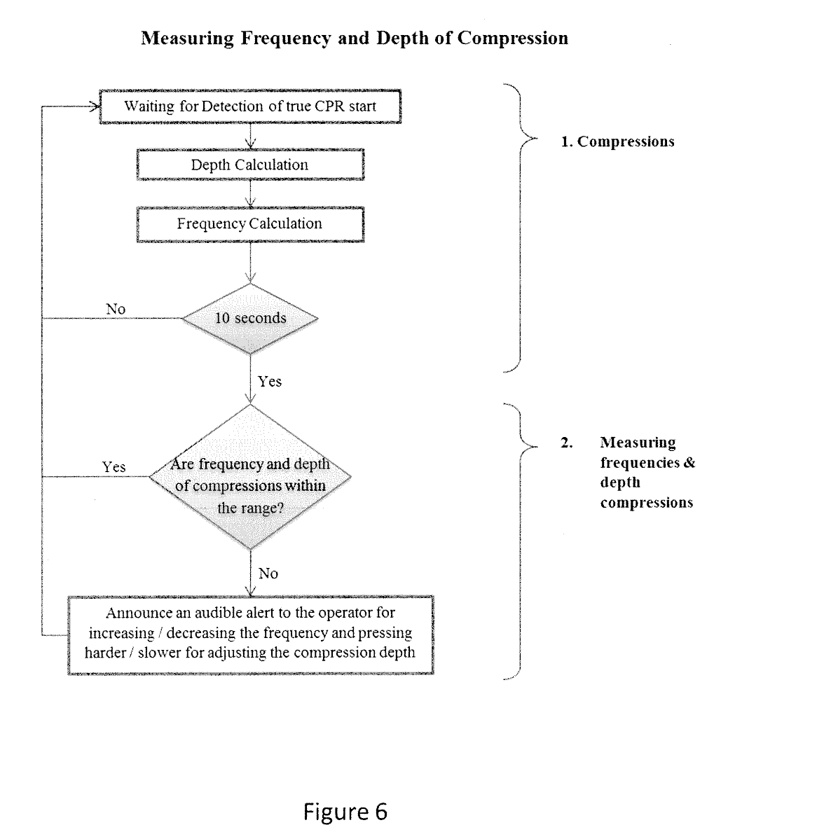

In one embodiment of the present disclosure, the sensors (including accelerometer) of the one or more smartphones are used to calculate the frequency and depth of compressions during CPR as shown in FIG. 6.

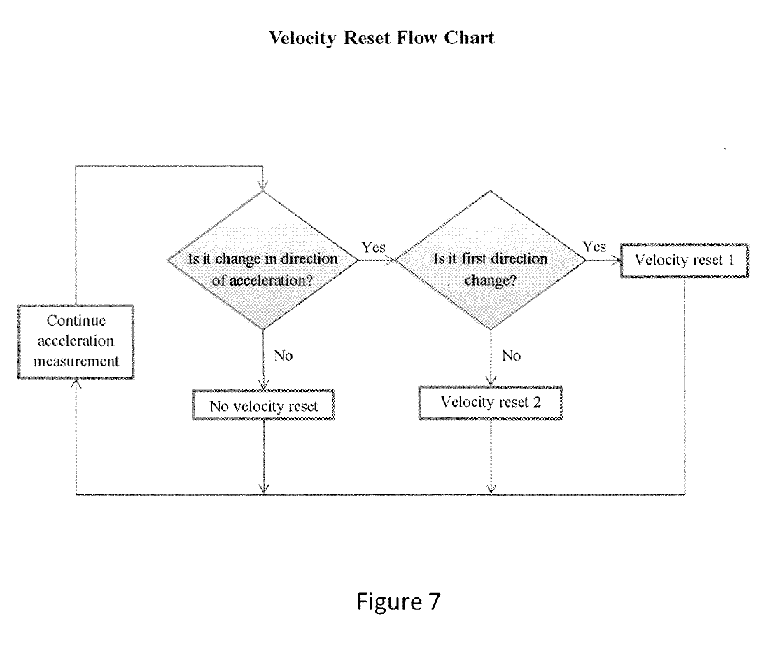

In one embodiment of the present disclosure, the sensors (including accelerometer) of the one or more smartphones can have the velocity reset to ground levels during measurements of the frequency and depth of compressions during CPR as shown in FIG. 7.
Set area=0
Get timerValue
Get Accelerometer Reading
Filter out Calculated noise by subtracting the noise parameter
If the accelerometer reading shows change in direction
   positive accelerometer reading is compression
   negative reading is recoil
   Report the area calculated until change in direction as velocity
If no change in direction then
   Dt=difference in time from previous Accelerometer reading
   Dacc=difference between current and previous accelerometer reading
Area=area+(Acce*Dt+(Dacc*Dt)/2)
Go to step 2.

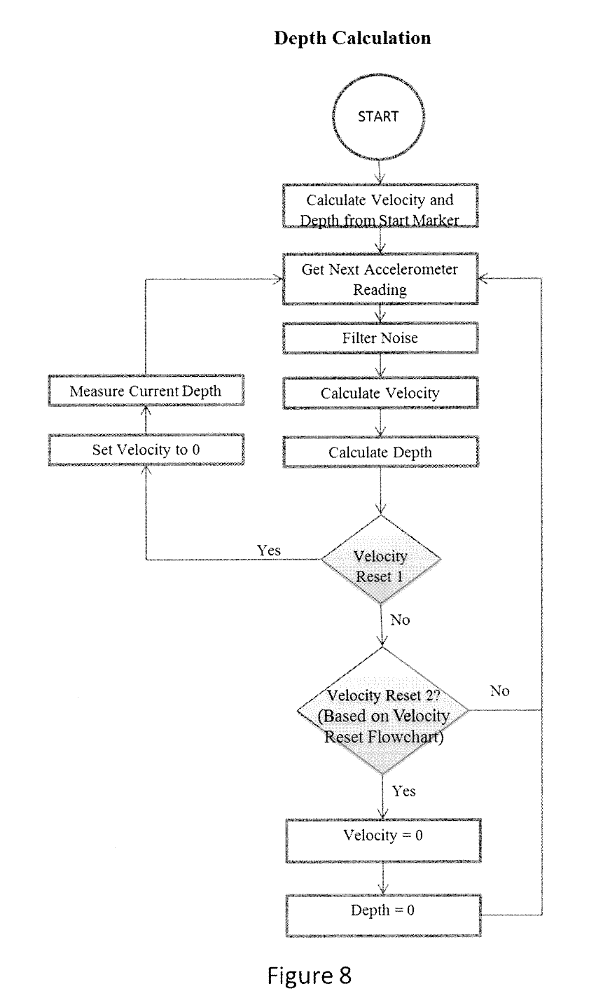

In one embodiment of the present disclosure, the sensors (including accelerometer) of the one or more smartphones are used to calculate the depth of compressions during CPR as shown in FIG. 8.
Set area=0
Get timerValue (i.e., the operating system function call)
Get Velocity calculated
   Velocity magnitude is positive when compressing
   Velocity magnitude is negative when compression recoils
If the velocity reading shows change in direction
   Report the area calculated until change in direction as depth
If no change in direction then
   Dt=difference in time from previous velocity
   Dvel=difference between current and previous velocity
   Area=area+(velocity*Dt+(Dvel*Dt)/2)
Repeat step 2.

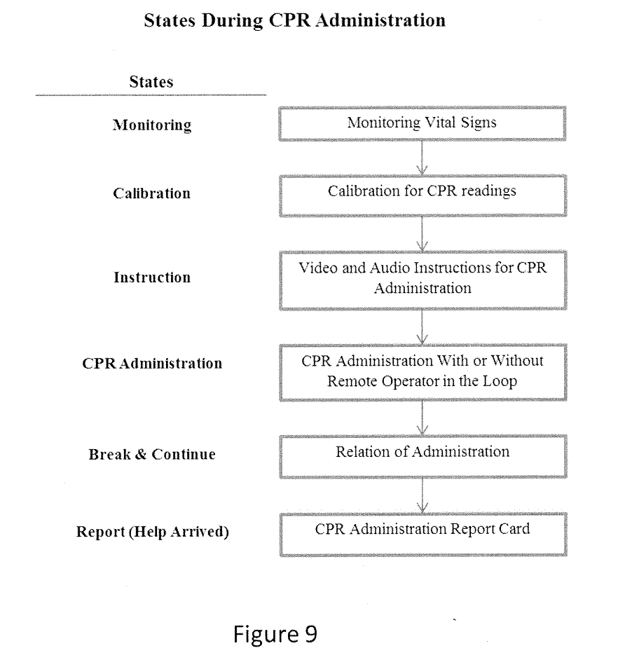

In one embodiment of the present disclosure, the one or more smartphones are used in multiple functions during the same or different phases during administration of CPR as shown in FIG. 9.

In one embodiment of the present disclosure, the method of administering CPR and monitoring vital signs can have one or more of the following features:
Ability to perform real-time feedback via the received vital signs sent to remote facility, logged in the smartphone, or a combination thereof;
Ability to provide real-time feedback from remote operator (e.g. 911 operator, CPR expert, or the like);
Ability to use the CPR administration method on real-life subjects or mannquins during CPR training;
Ability to provide rapid, impromptu audio and video instructions for administering CPR based on collected vital signs, preferred CPR methods, or a combination thereof;
Ability to provide periodic alerts for improving effective administration of CPR including desired compression frequency, compression depth, mouth-to-mouth breathing, or a combination thereof;
Ability to create dynamic frequency of alerts based on present performance of CPR based on measurements collected by the one or more smartphones;
Ability to calibrate the sensors based on the age, gender, size, and condition of the subject;
Ability to provide specific feedback about ideal CPR parameters based on the age, gender, size, and condition of the subject;
Ability to communication with remote operator using one or more smartphones during administration of CPR which another one or more smartphones continues to monitor the subject's vital signs;
Ability to generate alerts and feedback in various languages;
Ability to detect if every depression is fully recoiled before starting the next compression;
Ability to alert the user (i.e. CPR administrator) regarding ineffective CPR;
Ability to monitor chest compression fraction (CCF) and alert the user if there is more than about 20% rest during the CPR administration. CCF monitoring helps for effective rest periods during CPR administration and hence the administrator helps to continue CPR for long period of time. For example, CPR administration can be still effective if CCF is more than 80%. This means the administrator can pause for 15-20 seconds for every 2 minutes; and
Ability to generate a “report card” after the CPR administration is complete including CPR parameters such as chest compression fraction (CFF), compression frequency, compression depth, heart rate of the subject, oxygen saturation elvels of the subject, any other vital signs, or a combination thereof.

Issues and Source of Errors

Calculating depth using an accelerometer does not present a trivial task. It involves integrating acceleration readings to compute velocity and further integration of the calculated velocity to find the displacement or distance of movement. This error-prone process requires a sophisticated algorithm to determine the displacement. Several sources of error may arise in this process:
Errors inherent in the accelerometer or caused by noise from the electronic signal.
External errors—errors caused by force applied to the accelerometer such as lateral movements of the hands during CPR or movement in a vehicle when a patient is being transported while receiving CPR).
Error due to drift: these errors are introduced during the compression of the chest. For example, the chest may not fully recover to its normal position before the next compression is started. This drift results in the device reporting an incorrect starting position of the compression.

Unfortunately the process of double-integration on these readings compounds these accelerometer reading errors even a small error can produce large variation in calculated displacement.

Measuring blood oxygenation levels using a smartphone is even trickier because the method has to be non-invasive, should not require additional devices, and should be simple and quick for anyone to use, even if not a health professional.

Methodology for Measurement

This section discusses an example of the methods to measure the frequency and depth of chest compressions and the oxygen saturation levels using smartphones.

Frequency of compressions: An accelerometer measures acceleration of movement in the x, y, and z axes. When people lie on their backs, chest compressions are in the direction of the Z axis. So, each upward movement is considered to be negative acceleration; each downward movement is considered to be positive acceleration. A complete up and down movement counts as one compression. The frequency of compressions can be calculated as up-down movements.

Depth of compressions: The compression depth can be calculated using the acceleration measurement. The accelerator sends measurements to the smartphone's processor every few milliseconds. The processor, in turn, calculates the compression depth. A basic theoretical framework for measuring distance (depth) from acceleration is summarized below. Their straight-forward approach is to:
1. Calculate velocity from acceleration as follows: Given acceleration (a) and a period of time (t), it is possible to calculate the change in velocity during the relevant time period. If the original velocity is available, the change in velocity at the end of the time period can be calculated using the equation:

Δv=at
Where Δv is the change in velocity during time t. If the velocity at the start of the time 0 is v₀, then velocity at time t is:

v=v₀+Δv
2. Calculate the distance as follows:

$$Δ d = \frac{( {v}_{0} + v )}{2} * t$$

Where Δd is the change in distance, v₀ is the velocity at time 0, and v is the velocity at the time t. If d₀ is the distance at time 0, the distance at the time t is:

d=Δd+d₀

Unfortunately, these calculations assume a straight-line motion, which is not the case for CPR. CPR measurements, instead, resemble sine curves. Consequently, we require other methods to calculate displacement. More importantly, displacement needs to be calculated in real-time. This means velocity and displacement must be found while the accelerometer readings are still being logged, and so numerical methods must be used that allow for integration on data readings as they are logged. One method, trapezoidal rule, offers an approximation technique for calculating the integration.

The existing literature on calculating displacement from accelerometer readings is based on devices that are dedicated to CPR compressions. Dedicated devices can be calibrated accordingly. In most emergency situations special devices may not be easily accessible, but a smartphone with an accelerometer is more likely to be available. This example assumes that an untrained person administers the CPR and does not have access to such dedicated devices. Experimental data was not collected when CPR is performed on actual person. For obvious reasons it is not feasible to perform CPR on healthy people. Data is collected in CPR classes which use manikins that simulated chest compressions. For the examples, data was collected by placing the smartphone in the middle of the chest.

Blood Oxygen Saturation level: Blood Oxygen Saturation Levels were determined using the camera lens of the smartphone. Pulse oximeters measure the visible and infra-red spectrum of the oxy-hemoglobin and de-oxy hemoglobin, respectively. A pulse oximeter works by exploiting particular properties of light. When light passes through a substance (such as blood), the substance absorbs a certain amount of light. The amount of absorbed light depends on the sample's concentration, the sample's absorbance capacity, and the light's path length. The BOS level was calculated using Beer-Lambert's law:

A=abc
Where, A is absorption; a is molar absorptivity of the sample, c is concentration of the sample, and b is path length.

Oxy-hemoglobin (HbO2) and deoxy-hemoglobin (Hb) absorb light at different wavelengths. Oxy-hemoglobin (HbO2) absorbs more infra-red light than deoxy-hemoglobin (Hb); Hb absorbs more red light. The color red is in the spectrum 660 nm. The infrared spectrum has a wavelength of 820 nm-950 nm. Oxygen saturation is calculated using the following relation:

$$SpO 2 = f ( ϕ ) ; where φ = ( \frac{{AC}_{red} / {DC}_{red}}{{AC}_{ir} / {DC}_{ir}} )$$

### Experiments and Results

### A. CPR Experiments

Several experiments were conducted to collect data. This section discusses issues encountered during data collection and steps taken to resolve them.

One of the first problems resolved concerned placement of the smartphone on the person receiving CPR. CPR cannot be performed if the phone lies directly on the chest. Such a placement can be extremely uncomfortable and can cause injury if the smartphone is pressed against the chest during CPR. Placing the smartphone between the palms of the person giving CPR is also not feasible. The screen may crack under pressure. Additionally, this placement is uncomfortable for the person giving the CPR. The ideal solution was to place the phone above the hands of the person giving the CPR (see FIG. 2). In these experiments, the smartphone was tied to the hands so that it did not fall off. This can be done by any piece of cloth such as a shirt or undershirt.

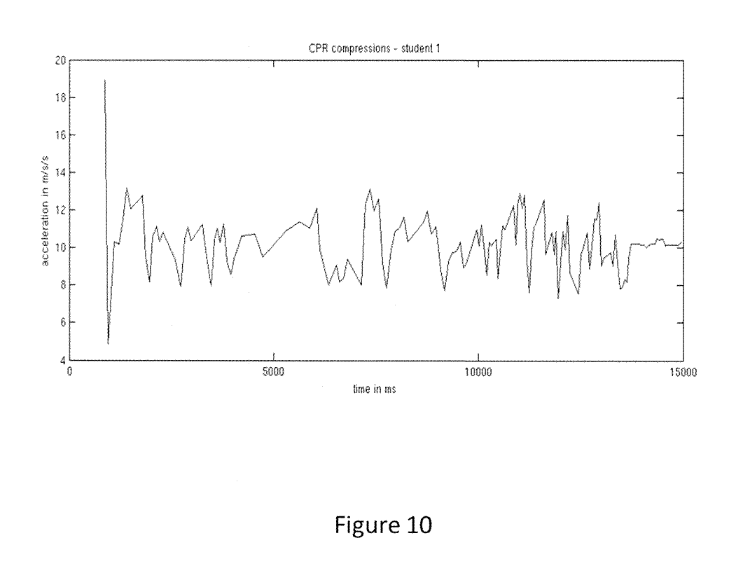

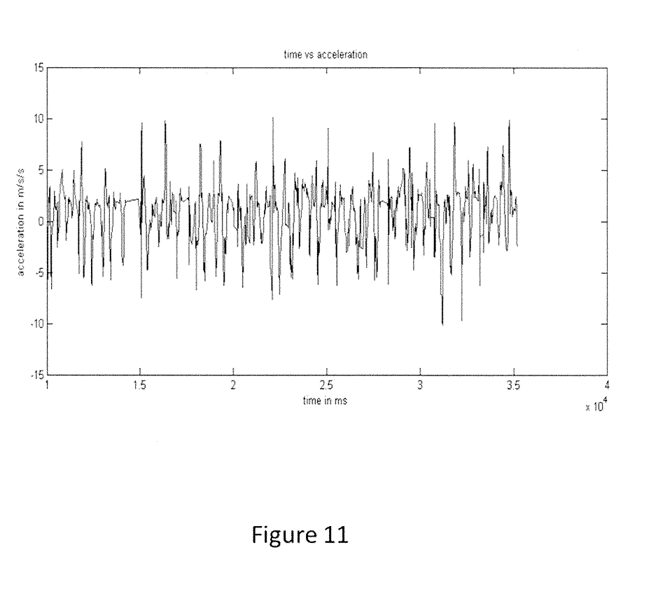

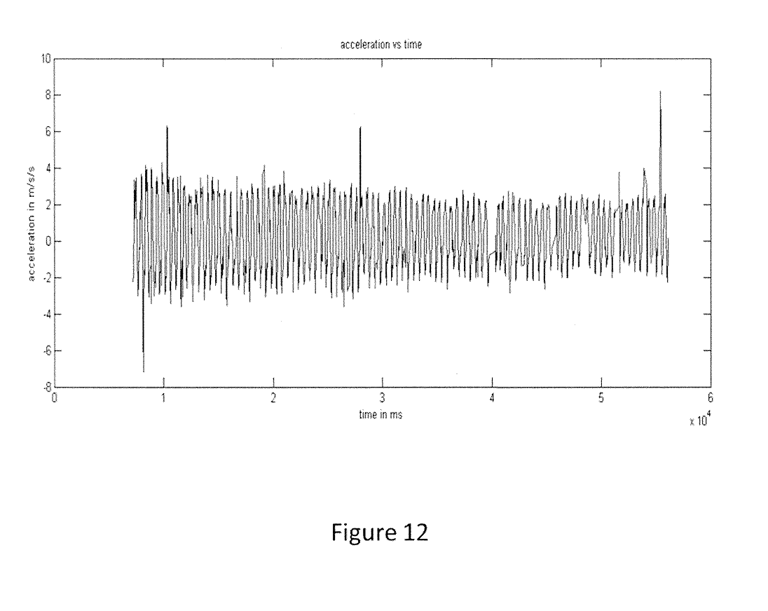

Another issue that needed to be resolved was which type of manikin to use. There are two kinds of manikins used for training—a soft-chest (sponge) manikin and a hard-chest manikin. FIG. 10 shows the accelerometer plot for a student performing CPR on a soft-chest manikin; FIG. 11 shows a similar plot for an instructor performing CPR on a soft-chest Manikin. As shown in both FIGS. 10 and 11, compression frequency and depth were not uniform over time. FIG. 12 shows the same instructor doing CPR on a hard-surface manikin. As shown in FIGS. 10-12, the best results were from the instructor doing the CPR on a hard-surface manikin. Specifically, FIG. 12 shows that compression frequency and depth were uniform over time, compared with the less consistent results in FIGS. 10 and 11. A hard Manikin was used to test the algorithm.

### B. Algorithm for Calculating Frequency and Depth of Compressions

Calculating the frequency of CPR compressions can be done with an accuracy of greater than 95%. When the accelerometer moves towards gravity, the acceleration is considered positive and when it moves against the gravity, it is considered as the negative direction. The number of times the accelerometer readings show a change in the sign of magnitude from positive to negative number can be counted.

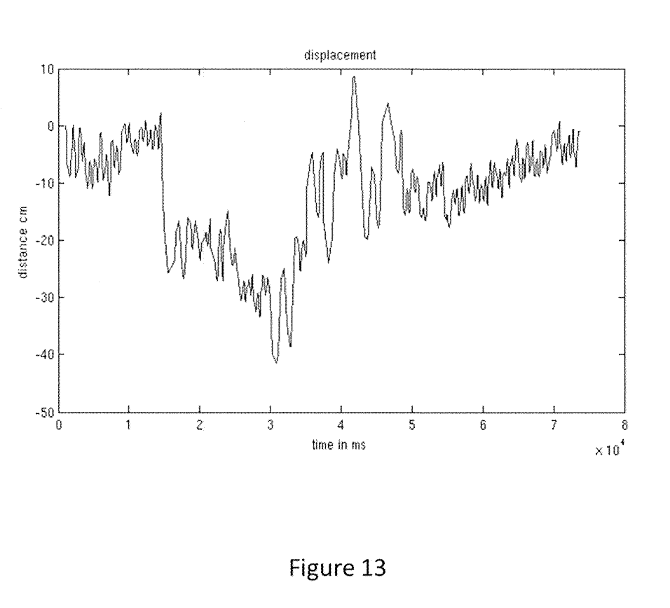

However, calculating depth of compression is subject to errors from several sources, and so the algorithm had to be fine-tuned to reduce the influence of such errors on our results. This section documents efforts to improve the accuracy of calculation of depth of CPR compressions. When a simple method of double integration was used to calculate the compression depth (see FIG. 13), the depth of compression varied up to 40 cm. The method needed correction to more accurately measure compression depth. As shown in FIG. 13, the calculated depth is out of range and errors accumulate over time.

The first issue to resolve is the granularity of accelerometer measurements. During the first attempt at collecting the CPR data, smartphone model Google-1 was used. This accelerometer logged in its readings with time gaps between tens of milliseconds and hundreds of milliseconds. As a result, the calculations derived from these readings were inaccurate and inconsistent. One of the reasons for this was the large time gap in the readings. The integration function uses the magnitude of acceleration and velocity over the time period between two successive readings. So, if the time between two successive readings was large, the algorithm calculated too large a displacement value. A more modern version of the smartphone [Samsung Galaxy] provided a higher granularity of readings, giving more than 100 readings per second. Furthermore, the readings are spaced at a more uniform time gap, increasing the accuracy of depth calculation. Two major changes were implemented in the basic algorithm to calculate the depth. The first change used the fact that CPR involves restricted and repeated movements in a vertical direction.

As mentioned earlier several sources can cause errors in displacement measurement. Even a minor error in the acceleration magnitude can result in a large error in displacement measurements. One way to reduce such errors employs calculating a rolling average of velocities. The algorithm was modified to calculate a rolling average after a number of accelerometer readings were logged.

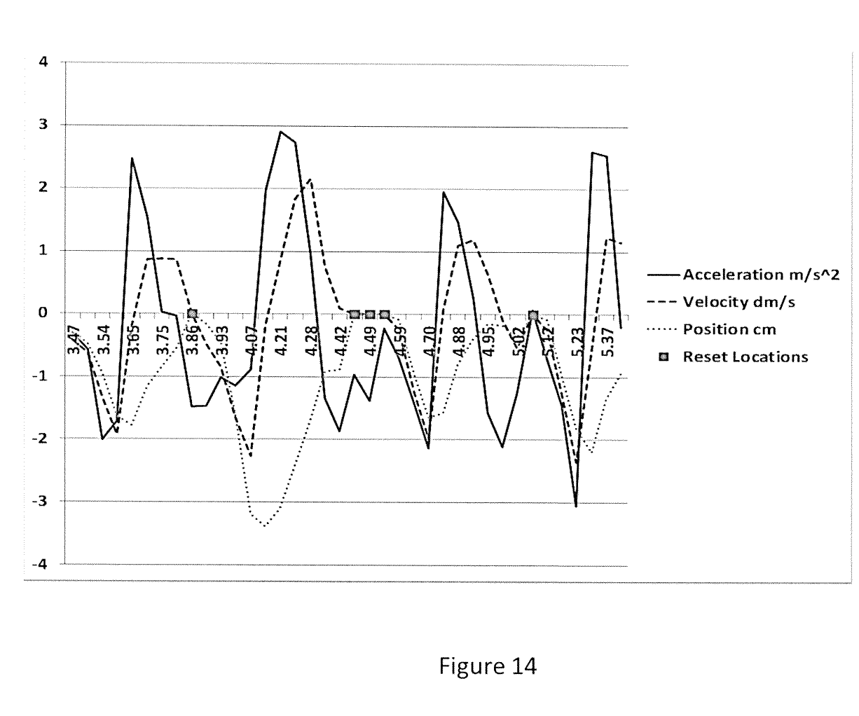

Finally, the following method to reduce the errors has proved to be the most promising. First, the fact that CPR movements are in vertical direction and are repeated motions within a range of 2-2.5 inches was taken advantage of. This fact was used to reset the calculated velocity to zero each time the motion changed direction. Note that compressions have zero velocity points twice during each chest compression—velocity is zero at the start of the compression, but the velocity also returns to zero when the chest, fully compressed, starts to return to its normal position. These two points were determined and the velocity reset to zero, even when the calculated velocity was not zero. This avoided error build up. Similarly, the depth calculation was reset to zero at the start of each compression, even though the calculated depth might be non-zero. FIG. 14 shows a plot of the acceleration magnitude and the corresponding velocity and the calculated distance. The bold dots show the points where velocity is reset to zero. The y-axis is m/s² for Acceleration, dm/s for Velocity and cm for distance. This solution localized errors of magnitude to within one compression, which provided further correction to errors that build over time. Particularly, this adjustment to the algorithm reduced error caused by drift.

### C. Accuracy of the Distance Calculation

In this section the method used to determine the accuracy of distance calculations is described. The experimental setup consists of the following steps:
Write an android application to calculate the CPR depth from the smartphone's accelerometer.
Use a commercially-available manikin designed for CPR training to collect data. The force required to press the manikin's chest must match the actual force required to press a human's chest during a real CPR.
Use a Mobotix camera to record CPR compressions. The professional security Mobotix camera allows the study of video frame by frame and determine actual depth of compression.
Compare the calculated CPR depth for each compression with the actual movement observed on video.

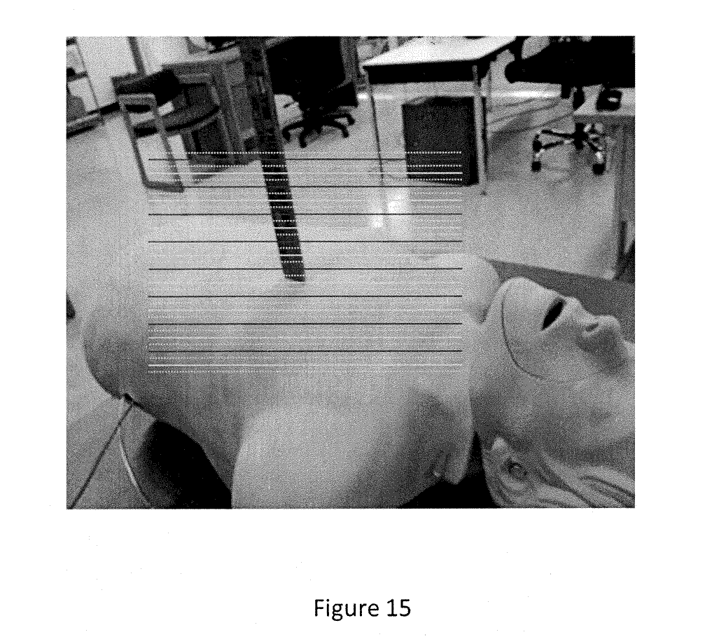

FIG. 15 shows the manikin used in our experiments. As shown in FIG. 8, a scale was overlaid on the image to allow us to calibrate the actual movement during CPR as compared to the observed movement on the video frame. As CPR was performed, the smartphone recorded the calculated depth in a file. The Mobotix camera recorded the CPR process in a file. After recording 40 seconds of CPR, the video file was played frame by frame and the actual depth of CPR was measured. The algorithm then compared the two to determine the difference between the actual depth of compression (as observed on the video frame) and the depth calculated by our application.

### D. Results

In this section experimental results are presented as well as a discussion of the application's accuracy of depth calculation. The application prompts the CPR giver to increase or decrease the depth of chest compression to meet the 2-2.5″ depth requirement. The accuracy with which the application provides alerts is also discussed. Similarly, the application gives a prompt when the frequency of compressions is not within a range of 90-100.

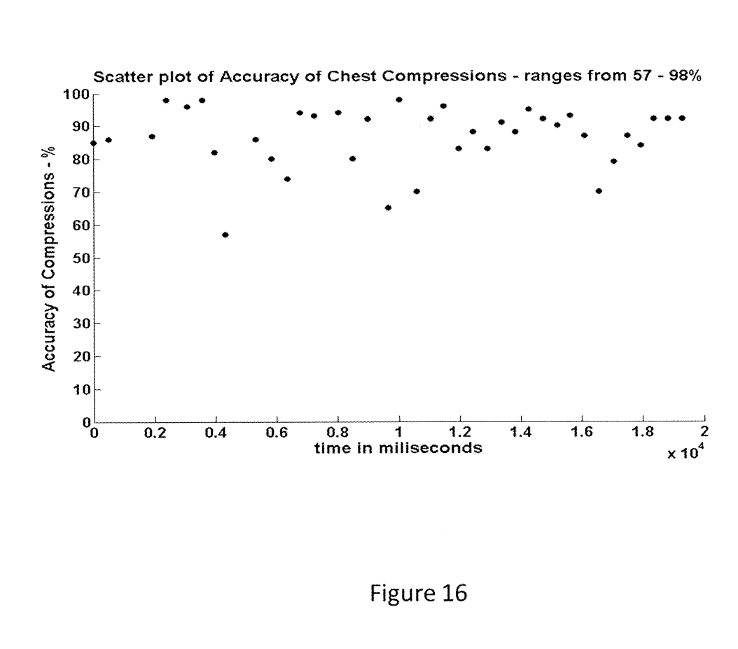

Experiments were conducted using 40 volunteers. Each volunteer performed CPR for about 30 seconds. The number of compressions volunteers performed during these 30 seconds ranged from 30 to 50. FIG. 16 shows a scatter plot of each compression for one subject. It shows the accuracy of the depth calculation done by the application as compared to the actual depth as observed in the Mobotix video. As shown in FIG. 16, the application's accuracy ranged from a low of 57% to a high of 98%. The other 39 subjects had similar ranges of accuracy.

### E. Accuracy and Frequency of Alerts

In the previous section the accuracy of the application to determine the depth of compression was discussed. The focus was on how accurately the application can calculate the depth as compared to the actual depth. This section focuses on the frequency and accuracy of giving alerts.

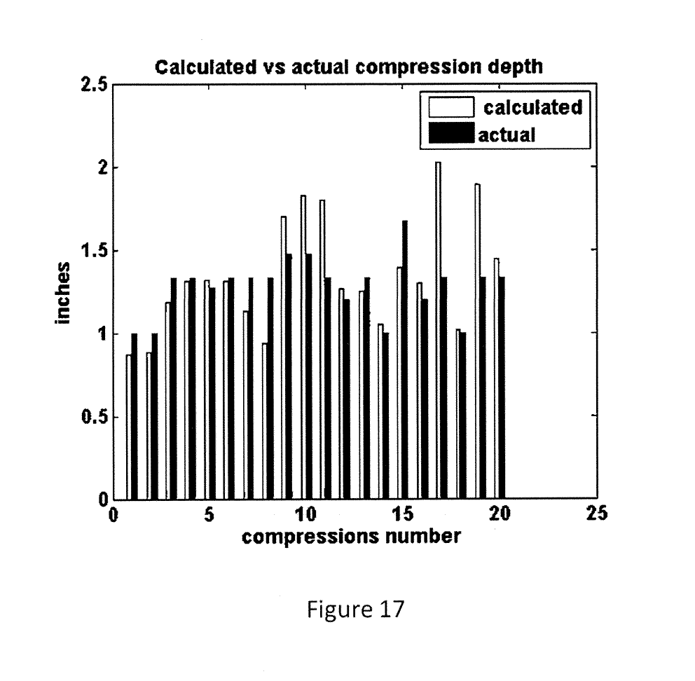

As has been noted earlier, the person administering the CPR must accurately judge whether the chest has been compressed to the recommended depth before releasing the chest to return to normal. The application gives an alert when the depth of the compression falls outside an acceptable standard range. One of the questions was “When should the application provide an alert?” One of the factors considered was the accuracy of the application's calculation. If the accuracy of a particular calculated depth of compression is low, then the decision to provide an alert by the application may be inaccurate. FIG. 17 shows a bar plot comparing the actual compression depth and the calculated compression depth for the same volunteer as in FIG. 16. It may be observed that the calculated depth value reaches near 2″ inch value for many compressions, but the actual depth never reaches the 2″ inch value. So, for these compressions, the algorithm will not give an alert, even though it should have given one. This issue was addressed by averaging the calculated depth over a period of time. Some of the calculated errors have a positive magnitude and some have a negative magnitude. So an average provides a better accuracy. Table-1 shows the accuracy of average over different time durations ranging from 1 second to 10 seconds.

A second factor is alert frequency. It is not feasible to alert for every compression. Too frequent alerts can overwhelm the person giving CPR. A person requires a few seconds to understand and respond to an alert. By the time the person reacts, another alert may already have sounded. This leads to confusion and the person may not be able to adjust their compressions accurately and in a timely manner.

An analysis was done to decide how frequently the alert should be given (Table 1). When the application gave an alert for each compression, out of a total of 38 alerts (for 38 compressions in that session), 21 alerts were less than 90% accurate. This means that for 21 alerts the accuracy of calculated depth as compared to the actual depth was less than 90%. When the application gave an alert every second, the calculated depth of all compressions was averaged within each second. As Table 1 indicates, the total number of alerts for one second will be 20. Of these 9 alerts will be less than 90% accurate. When the application gave an alert every 6 seconds, then 4 alerts were more than 90% accurate. This analysis continued through 10 seconds. At 10 seconds, the application gave two alerts, each 100% accurate. The accuracy of alerts increases when an alert is given every few seconds rather than for every compression. The accuracy improves because the errors with negative magnitude adjust the errors with positive magnitude with in the time period. So the overall accuracy improves. Within the 6-7 second range, the application's accuracy is reasonable at more than 90%. But, experiments suggest an alert every 6-7 seconds does not provide persons giving CPR enough time to adjust their CPR compression depth. Experiments also suggest that an optimum time for giving alerts is every 10 seconds.

TABLE 1
Accuracy of Alerts over Time

| Frequency | Total | Accuracy Range for Alerts - % | | | | |
|---|---|---|---|---|---|---|
| of Alerts | Alerts | <80 | 80-85 | 86-90 | 91-95 | 96-100 |
| each comp | 38 | 6 | 7 | 8 | 12 | 5 |
| 1 second | 20 | 3 | 3 | 3 | 8 | 3 |
| 2 seconds | 10 | 0 | 0 | 3 | 4 | 3 |
| 3 seconds | 7 | 0 | 0 | 3 | 2 | 2 |
| 4 seconds | 5 | 0 | 0 | 1 | 3 | 1 |
| 5 seconds | 4 | 0 | 0 | 1 | 2 | 1 |
| 6 seconds | 4 | 0 | 0 | 0 | 4 | 0 |
| 7 seconds | 3 | 0 | 0 | 0 | 1 | 2 |
| 10 seconds | 2 | 0 | 0 | 0 | 0 | 2 |

Referring now to Table 1, the rows show accuracy for alerts given for each compression, and for each second between 1 and 10 seconds. Frequency of Alerts shows the time period analyzed. Total Alerts shows the total number of alerts that occur during the specified time period. Accuracy Range for Alerts—% shows, in 5% increments, the number alerts with compression accuracy. For example, when an alert is given every second, 6 alerts of the 20 alerts have a compression accuracy of less than 85%. However, when an alert is given every 5 seconds, all the alerts have compression accuracy greater than 85%.

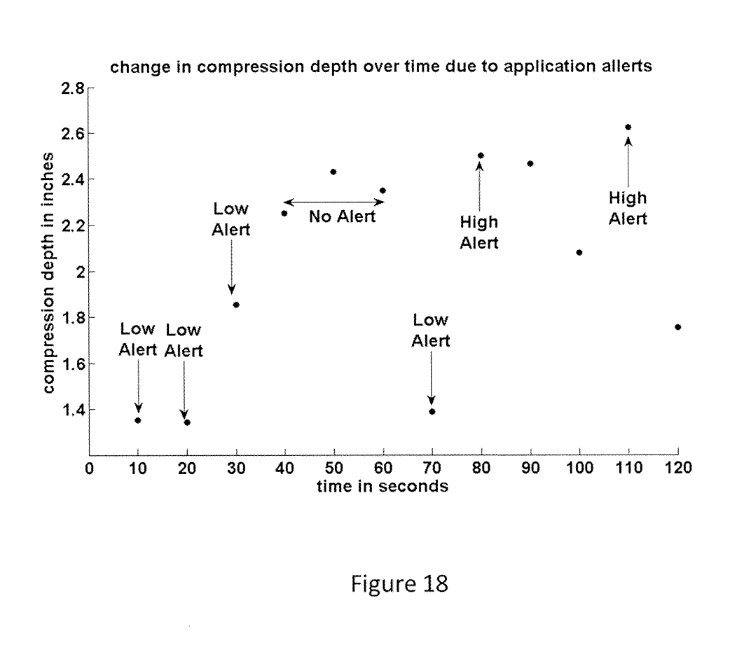

As explained, alerts provide feedback to a person giving CPR so that person can adjust the compression depth or frequency to fall within a prescribed range. FIG. 18 shows a scatter plot of the compression depth for one subject. The duration of CPR session depicted in this plot was 120 seconds. The application issued alerts when the compression depth fell below 1.5″ (Low Alert) or rose above 2.5″ (High Alert). Low Compression alerts indicate compression depth should be increased. High Compression alerts indicate compression depth should be decreased. Initially, the compression depth was 1.4″. The application provided a Low Alert at 10 seconds and then, again, at 20 seconds. At 30 seconds, compression depth increased to 1.8″ and the application gave another Low Alert. The compression depth then increased to more than 2″ for a 30-second period, so no alerts were issued. At 80 seconds, the depth was greater than 2.5″, so our application provided another alert. It was concluded that the application achieved its purpose of providing alerts to the CPR giver, which enabled the CPR giver to adjust to more effectively administer CPR.

Table 2 shows the overall compression depth accuracy of CPR sessions for all participants. The results show that the accuracy ranges from a low of 77% to a high of 99%. The average accuracy is 93.8%. But most readings are more than 90% accurate. Only 3 people had an accuracy of less than 90%.

TABLE 2
Accuracy of Depth Calculation for All 40 Participants

| Average % | Minimum % | Maximum % | Median % |
|---|---|---|---|
| 93.8 | 77 | 99.6 | 88% |

### F. CPR in a Moving Vehicle

In certain situations, one may have to give CPR as the patient is being transported in a moving vehicle to the hospital. Several factors come into play in a moving vehicle that increases the difficulty of calculating the compression depth accurately when using a smartphone accelerometer. The first, a vehicle moving affects accelerometer readings. If shock absorbers are inadequate, an accelerometer records vehicle movements in the Z axis, skewing the Z axis motion of the chest compressions. Road condition presents another major factor that contributes to increased errors. Bumps in the road, lane changes, and traffic turns also affect readings. Traffic patterns also add to the randomness of readings. The vehicle may have to be slow at times and then accelerate as the traffic moves. It may have to stop at traffic lights and then accelerate. Even if factors are kept constant, such as using the same vehicle, driving on the same road and even driving at the same speed, randomness of a traffic pattern still produces different results each time the CPR is attempted.

An experiment was conducted with extremely controlled conditions. A smooth road with no bumps was selected. The road had almost no traffic, required no turns for a few miles, and had no traffic lights. The car was driven at a constant speed of 30 mph to minimize movements due to vehicle motion. The results of the experiments are shown in the Table 3. The high standard deviation indicates that there was large variation in compression depths. This was caused by driving conditions and the vibrations of the vehicle itself. However the frequency calculations were accurate.

TABLE 3
Results of CPR in a Moving Vehicle, Showing the Depth
of Compressions in Inches

| Average Compression | Median Compression | Standard Deviation |
|---|---|---|
| 1.721 | 1.664 | 1.085 |

### G. Calculation of Oxygen Saturation of Blood

CPR's purpose is to circulate oxygen-carrying blood through the body. Should the injured person's Blood Oxygen Saturation Level drop precipitously, the person may suffer physical deterioration and even death. To reduce this risk, the algorithm must monitor the BOS levels while it is measuring compression frequency and depth. In this section a procedure is described that uses smartphones to measure the BOS level using principles of optics. While a person gives CPR, this information can assist in deciding whether to give breathing. Mouth-to-mouth breathing replenishes the oxygen in the blood stream. However, many prefer to avoid using the technique unless absolutely necessary because of possible exposure to infectious diseases. The algorithm needed to provide a method for the 9-1-1 dispatcher or the person giving the CPR to determine whether mouth-to-mouth breathing was really necessary. To resolve this issue, a process was devised that makes use of the smartphone's optical capabilities. The surface of the injured person's fingertip (the area of analysis) is placed on the smartphone's camera lens. By taking a video, a beam of near-infrared light is passed through the finger. As the light passes through, the smartphone creates video of the area of analysis. The video is then analyzed to determine the RGB values (red green blue). RGB values of the refracted light in the blood are then analyzed for the scattering effect of near-infrared light. This scattering effect allows determination the BOS level.

Validation of Low Oxygen Levels:

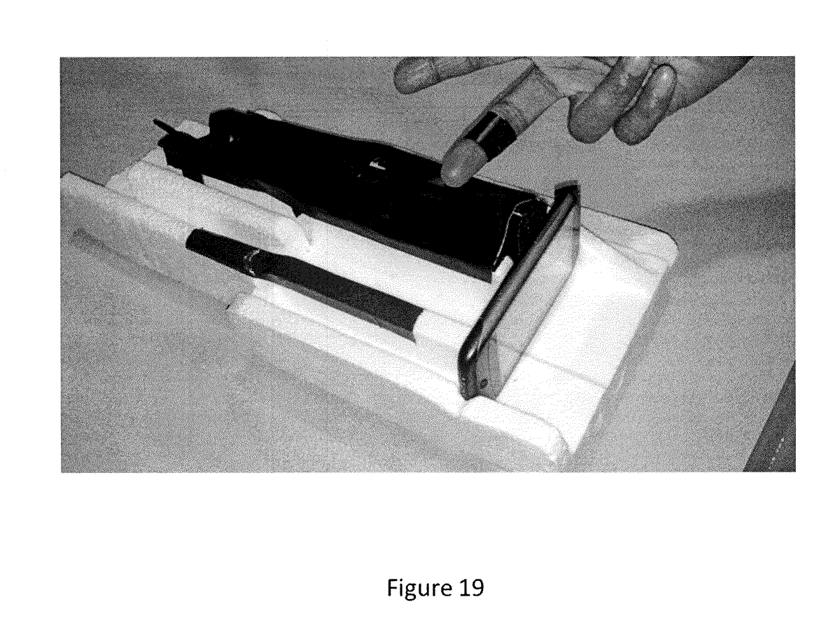

After measuring the Oxygen saturation level under normal circumstance, a confirmation that the system can detect reduced levels of oxygen in the blood was needed. Occlusion spectroscopy, the method of using over-systolic pressure to temporarily stop the flow of blood to the finger to collect data, was used (FIG. 19). In the study, the blood flow in the root of the finger was temporarily cut off. The blood flow to the finger is decreased by applying pressure to it. This allowed the measurement of blood flow in the upper layers of the finger. Later when occlusion was removed, the oxygen saturation level reached the normal value and the system can measure this return to the normal value. The occlusion experiment confirmed that the depletion of oxygen saturation in the blood can be measured using the optical features of a smartphone. In fact, occlusion allowed a set of data points instead of one data point.

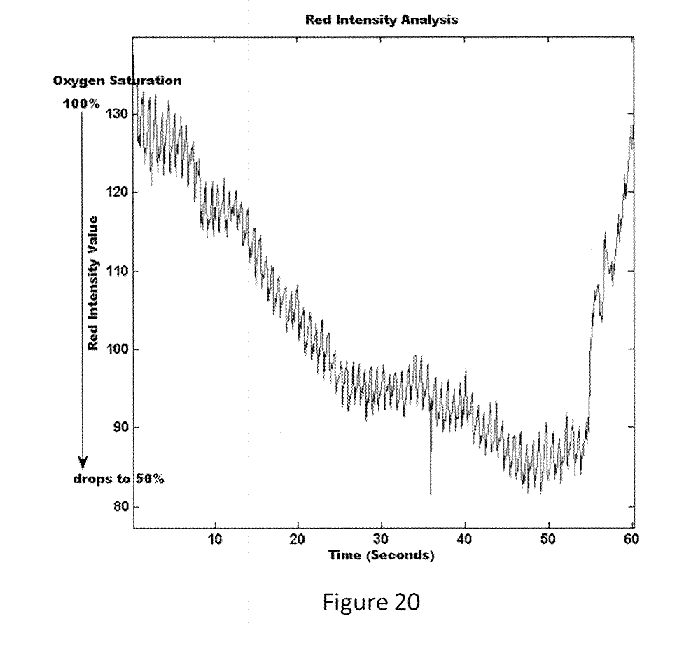

To establish standardization, a method of dividing the intensity of the blood pulse (AC) by the intensity of the blood color (AD) was needed. This was applied to data to analyze both the intensity of green and of red values. The green AC/DC value was then divided by the red AC/DC value. This value corresponds to the percentage of blood oxygen saturation. FIG. 20 shows the oxygen saturation level as it drops to 50% after occlusion. The graph also shows that the oxygen saturation level recovers back to 100% after occlusion is removed. FIG. 20 shows a drop in oxygen saturation level to about 50%, but then the saturation level returns to 100% after occlusion is removed.

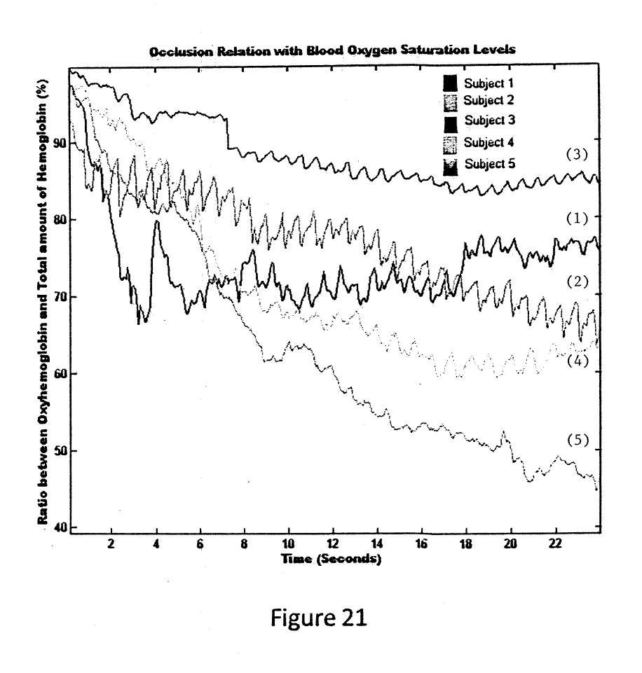

As time passes with occlusion, a decay of oxygen occurs. Through experimentation, it was found that the rate of the decay of oxygen varies with each subject. While some subjects experienced a quick decrease of blood oxygen, other subjects experienced a slow decay. This decay for 5 subjects is shown in FIG. 21. As shown in FIG. 21, the amount of oxygen within the blood in the finger dropped significantly for the first few seconds and then finally stabilized for each subject.

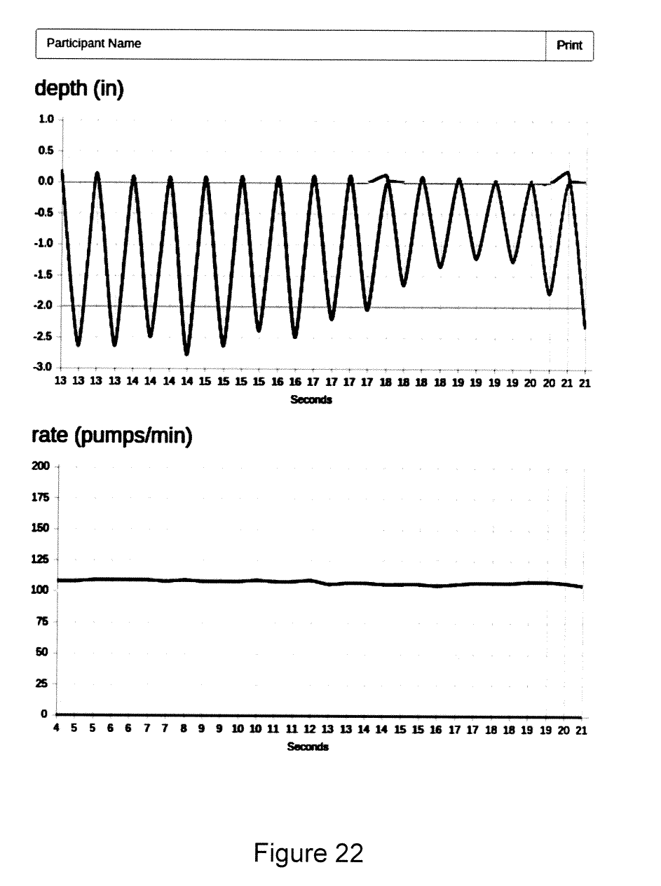

FIG. 22 shows the appearance of at least portions of an example NG911 operator portal that may be used in accordance with exemplary embodiments.

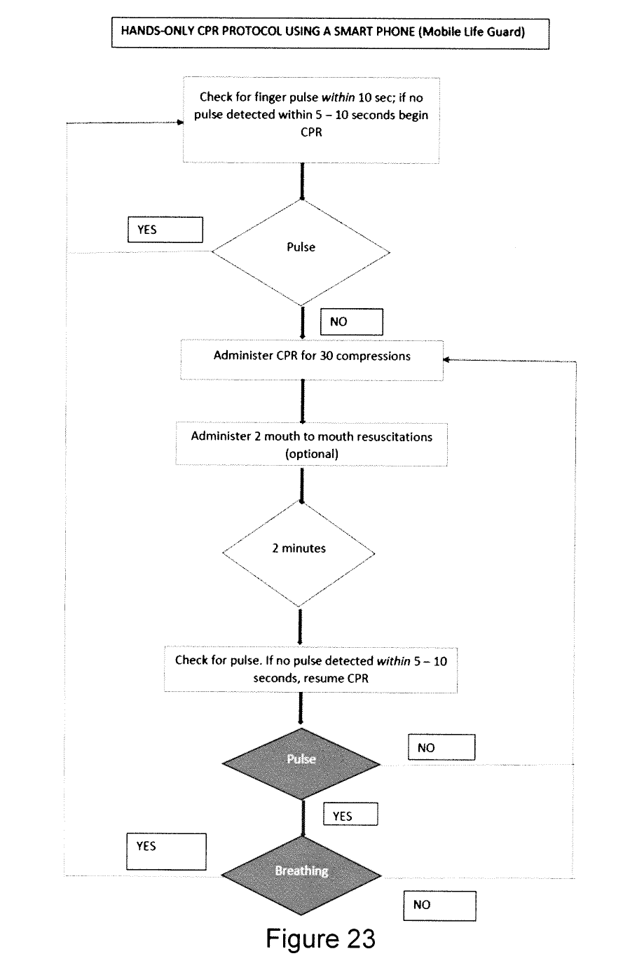

In another embodiment of the present disclosure, hands-only CPR is administered to unresponsive subjects or mannequins during CPR training using the algorithm shown in FIG. 23. This embodiment involves the use of compressions without breathing. This protocol is considered to be complaint with the existing AHA guidelines If there are two bystanders, one person can check the pulse immediately after each 2 minute compression cycle when compressions have momentarily stopped. If the bystander is not interested in the mouth-to-mouth resuscitations, then it is acceptable to skip this event. All the measurements (CPR frequency, depth, pulse, breathing) are transmitted/logged to the remote web server. If the victim is obese or big, then deeper compressions of more than 2 inches are advised. If CPR is continued even after the pulse is observed, then there is a danger of breaking the ribs. The application also alerts the person administering CPR that the compression is not recoiled well.

### CONCLUSIONS

The advantages of timely CPR have been well recognized in the medical community. There are programs in place to teach people how to administer effective CPR. But, in emergency situations, a trained person may not be available. Our application uses a smartphone to evaluate the CPR being given. The application can then provide feedback to the person administering CPR to improve its effectiveness. Existing applications available on smartphones simply furnish a short video tutorial on how to perform CPR. Our smartphone application prompts the CPR giver in real time on when and how to adjust their frequency and depth of chest compressions to meet CPR guidelines. Our experiments' results show that our smartphone application can be used to effectively administer CPR, even by people who have not been trained to give CPR [32]. In emergency situations, where a trained person may not be easily available and timing is crucial, these devices can mean the difference between life and death. Additionally, the devices' sensors can also help by continuing to provide vital information to paramedics as they rush a patient to a hospital. By measuring oxygen decay using the smartphone camera as occlusion is induced upon the finger, our application allows accurate determination of the blood oxygen saturation level. By using the ubiquitous smartphone, people performing cardiopulmonary resuscitation can also determine when the frequency and depth of their compressions enhance blood flow. For example, the oxygen saturation level may offer a better indicator of CPR effectiveness than the depth or frequency of compressions. This also improves the CPR procedure for the trained people. They can determine when to provide mouth-to-mouth breathing.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A method for performing CPR, comprising:
activating an application on one or more mobile phones having one or more sensors;
placing one or more mobile phones on the finger of a subject to collect information about the subject;
determining whether CPR is necessary based on the collected information about the subject;
calibrating the sensors of the one or more mobile phones;
placing the one or more mobile phones in a position on a hand of a user of the one or more mobile phones;
administering chest compressions to the subject;
activating a sensor of the one or more mobile phones, including an accelerometer sensor, to permit the application to capture information about the chest compression rate and displacement relating to movement of the chest of the subject; and
transmitting the chest compression rate and displacement information of the subject to the emergency dispatcher using the mobile phone.

2. The method of claim 1, wherein the application utilizes Photo Plethysmography (“PPG”) to capture information about the heart rate of the subject relating to light variations in the finger tip of the subject.

3. The method of claim 1, wherein the activating the flash of the camera step occurs more than once.

4. The method of claim 1, wherein the one or more mobile phones is placed in a horizontal position on the hand of the user.

5. A method for transmitting overall health information of a subject to an emergency dispatcher, comprising:
activating an application on one or more mobile phones having one or more sensors including a camera having a flash;
placing one or more mobile phones on the finger of a subject to collect information about the subject;
activating the flash of the camera of the one or more mobile phones to permit the application to capture information about the blood oxygen levels, heart rate, of a combination thereof, of the subject relating to light variations in the finger tip of the subject;
transmitting the captured information of the subject to the emergency dispatcher using the one or more mobile phones;
placing the one or more mobile phones in a position on a hand of a user of the one or more mobile phones; and
administering chest compressions to the subject.

6. The method of claim 5, wherein the application for estimating heart rate information utilizes Photo Plethysmography (“PPG”) to capture information about the heart rate of the subject relating to light variations in the finger tip of the subject.

7. The method of claim 5, wherein the activating the flash of the camera step occurs more than once.

8. The method of claim 5, wherein the one or more mobile phones is placed in a horizontal position on the hand of the user.

9. The method of claim 5, wherein the camera of the mobile phone is capable of capturing still or video images and further comprising using the camera of the one or more mobile phones to transmit still or video images of the subject to the emergency dispatcher.
